(19) United States
(12) Patent Application Publication
(10) Pub. No.: US 2010/0124578 A1
(43) Pub. Date: May 20, 2010

(54) Appetite-suppressing weight management composition

(76) Inventors: Marvin A. Heuer, (US); Kent Clement, (US); Megan Thomas, (US); Jason Peters, (US)

Correspondence Address: IOVATE HEALTH SCIENCE RESEARCH INC., 381 North Service Road West, Oakville, ON, L6M 0H4, CA

(21) Appl. No.: 12/313,126

(22) Filed: Nov. 17, 2008

(51) Int. Cl. A61K 36/87; A61K 36/24; A61P 3/04

(52) U.S. Cl. 424/766; 424/725

(57) ABSTRACT

Compositions and related methods directed at supporting weight loss. Compositions combining at least one appetite suppressing ingredient with at least one other ingredient for maintaining or reducing body weight are provided. Additional embodiments provide for more than one ingredient with appetite suppressing activity combined with at least one other ingredient for maintaining or reducing body weight.

### FIELD OF THE INVENTION

The present disclosure relates to compositions and methods for reducing or maintaining body weight in a subject. The compositions contain a combination of ingredients directed at reducing or maintaining body weight, including appetite suppressing ingredients.

### BACKGROUND OF THE INVENTION

Obesity has become an increasingly widespread and predominant health concern. According to the World Health Organization (WHO) obesity is considered a multifactorial chronic disease which is increasing in frequency. Obesity, a condition of excessive body fat, generally results from more energy (food) being consumed than is being used. Stemming from excessive body fat, several health-related concerns such as increased morbidity have been linked to obesity and being overweight as well as hypertension, coronary heart disease, type 2 diabetes mellitus, stroke and even some forms of cancer.

Not surprisingly, a great deal of effort has gone into addressing the problems created by excessive weight. Fad diets, diet pills, diet foods, liposuction, intestinal bypass surgery, and lifestyle changes have all been advanced as possible solutions to the problem of weight loss in overweight individuals, some with greater success than others.

Given the prevalence and serious problems associated with obesity, and the significant drawbacks associated with intrusive surgical procedures and the lack of success of fad diets, a need exists for a high-compliance weight loss composition that is easy to administer to persons that are overweight or obese to reduce weight gain, cause weight loss, and reduce body mass index to acceptably healthy levels.

### SUMMARY OF THE INVENTION

The present disclosure describes methods and compositions for reducing or maintaining body weight in a subject. Compositions are disclosed which contain an ingredient directed towards suppressing the appetite of a subject in addition to another ingredient for reducing or maintaining body weight with activity other than, or in addition to, suppressing the appetite. Additional embodiments contain more than one ingredient directed towards suppressing the appetite of a subject in addition to another ingredient for reducing or maintaining body weight with activity other than, or in addition to, suppressing the appetite.

Further embodiments contain more than one ingredient for reducing or maintaining body weight with activity other than, or in addition to, suppressing the appetite in addition to one or more ingredient directed towards suppressing the appetite of a subject. The present disclosure further describes like-directed methods utilizing the disclosed compositions.

Thus, in certain embodiments, the present invention provides methods of maintaining or reducing the body weight of a subject by providing a composition comprising an appetite-reducing compound, and an additional compound with activity conducive to maintaining or reducing weight other than, or in addition to, appetite-reducing activity. The appetite-reducing compound may be a Caralluma species or a Hoodia species and the additional compound may be selected from the group consisting of Cissus quadrangularis, methylxanthines, Morus alba, chromium polynicotinate, hydroxycitric acid, and flavanols.

In other embodiments, the invention comprises compositions which comprise an appetite-reducing compound, and an additional compound with activity conducive to maintaining or reducing weight other than, or in addition to, appetite-reducing activity. In certain embodiments, the appetite-reducing compound is Caralluma fimbriata and the additional compound is Cissus quadrangularis. In other embodiments, the appetite-reducing compound is Caralluma fimbriata and the additional compound is a methylxanthine, Morus alba, chromium polynicotinate, hydroxycitric acid or a flavonol.

In still other embodiments, the invention comprises compositions which comprise an appetite-reducing compound, and an additional compound with activity conducive to maintaining or reducing weight other than, or in addition to, appetite-reducing activity. In certain embodiments, the appetite-reducing compound is Hoodia gordonii and the additional compound is Cissus quadrangularis. In other embodiments, the appetite-reducing compound is Hoodia gordonii and the additional compound is a methylxanthine, Morus alba, chromium polynicotinate, hydroxycitric acid or a flavonol.

In further embodiments, the invention comprises compositions comprising Caralluma fimbriata, Hoodia gordonii, and Cissus quadrangularis. The compositions may further comprise at least one of: a methylxanthine, Morus alba, chromium polynicotinate, hydroxycitric acid, or a flavanol.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present disclosure is directed towards compositions and methods for maintaining or reducing body weight in a subject by appetite reduction and other mechanisms conducive to maintaining or reducing body weight.

As used herein, the phrase “maintaining or reducing body weight” refers to any action, process or intervention that acts towards or has the effect of opposing, inhibiting, preventing or in any way lessening body weight gain. Interventions that are known for maintaining or reducing body weight include, but are not limited to: increasing thermogenesis, use of lipase inhibitors (inhibit fat metabolism and absorption), use of carbohydrate blockers (inhibit carbohydrate metabolism), and use of appetite suppressants. It is herein understood that body weight is made up of different components such as lean tissue (muscle, bone and organs) and non-lean tissue (fat). The component of body weight that is preferably maintained or reduced is fat.

As used herein, the term ‘subject’ refers to mammals and non-mammals. Mammals refers to any member of the Mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like.

As used herein, the term “derivative” refers to a compound, such as a salt, ester, or amine, which can readily supply a closely related biologically active compound, either upon administration or upon exposure to specific environmental conditions, such as pH, temperature, etc. For example, a “derivative” of hydroxycitric acid can be a salt, ester or amine of hydroxycitric acid, so long as the derivative can readily supply biologically active hydroxycitric acid under physiological conditions. For a given compound, the skilled person in the art will readily recognize and envisage those closely related compounds which should be considered “derivatives.”

Thus, for example, a derivative of a particular substance may comprise a form of that substance which has been modified through reaction. Other derivatives are forms of a given substance that are precursors of that substance which would give rise to that substance after modification. Reactions involved in the formation of derivatives include, but are not limited to: hydroxylation, esterification, amide formation and salt formation.

The compositions according to the present disclosure may be included in foods, dietary supplements, nutraceuticals, medical foods, botanical drugs, homeopathic remedies, over-the-counter drugs, prescription drugs, and compounded drugs.

The acceptable routes of administration compatible with the various embodiments of the present disclosure include those well-known in the art and include: oral, rectal, and parenteral. As used here, the term ‘parenteral’ refers to methods of administration to that region outside of the digestive tract. Examples of parenteral routes of administration include, but are not limited to, subcutaneous, intramuscular or intravenous injection, and nasopharyngeal, mucosal or transdermal absorption. The preferred route is administration is oral and includes acceptable oral dosage forms commonly known in the art.

As used here, the term ‘acceptable oral dosage form’ would be known by one of skill in the art to include, for example, powder beverage mixes, liquid beverages, ready-to-eat bars, hard and soft capsules, tablets, caplets, and dietary gels.

Furthermore, the dosage forms of the present disclosure may be provided in accordance with customary processing techniques for any of the forms mentioned above. Additionally, the compositions set forth in the example embodiments herein may contain any appropriate number and type of excipients, as is well known in the art.

Material of the present disclosure that is of plant origin may be in the form of an extract. An extract, as used herein, is most simply a preparation derived from a plant source. Extracts suitable for use in the present disclosure may be produced by extraction methods as are known and accepted in the art such as alcoholic extraction, aqueous extractions, carbon dioxide extractions, for example. Extracts may be concentrated, removing most of the solvent and/or water. Such extracts are typically liquid but may subsequently be provided as a dry powder. Plant extracts may be standardized to a known compound present in the extract.

A plant extract may be made from the entire plant or any part thereof. Plant parts include leaves, stems, flowers, inflorescences, shoots, cotyledons, etc. The various parts may be dehydrated or used flesh. Often, the plant parts are washed before processing. Alternatively, forms of unprocessed, or raw, plants may be used in embodiments of the present disclosure. Such forms may be whole or part and may be fresh or dried. In preferred embodiments of the present disclosure, plant extracts are used.

Caralluma

Caralluma is a cactus-like plant that grows abundantly in areas of Asia, the south Mediterranean, and parts of Africa. Caralluma is a cactus that has historical use as an appetite suppressant during times of famine in India and Africa. In India, the plant is boiled and eaten whole but elsewhere only the green follicles are consumed. The name of the plant varies between regions and in addition to Caralluma, it is also known as: Ranshabar, Maked shenguli, and Shindala makad.

Caralluma is reported to have anti-inflammatory, anti-nociceptive, anti-hyperglycemic, anti-ulcer, cytoprotective and appetite suppressant properties. In addition to the anti-hyperglycemic and appetite suppressant properties, Caralluma may have other activities conducive to maintaining or reducing weight such as the blocking the activity of the fat synthesis enzyme, citrate lyase. The phytochemical constituents of Caralluma include various glycosides (pregnane, flavone and megastigmane glycosides), bitter principles, saponins and various flavanoids.

There are a number of species of Caralluma including but not limited to: C. fimbriata, C. indica, C. attenuata, C. tuberculata, C. edulis, C. adscendens, C. stalagmifera, C. umbellata, C. penicillata, C. russeliana, C. retrospiciens, C. arabica and C. lasiantha.

It is herein understood by the inventors that inclusion of Caralluma in a composition will support the maintenance or reduction of body weight in a subject by at least reducing appetite. It is further understood by the inventors that Caralluma may further support the maintenance or reduction of body weight in a subject by antagonizing fat synthesis. Embodiments of the present disclosure contain Caralluma plant material as set forth in the examples below. The preferred species of Caralluma is C. fimbriata. The preferred form of Caralluma plant material is an extract from the aerial parts of the plant.

Hoodia

Hoodia is a cactus native to Africa that has been used traditionally to ease hunger discomfort and is currently being marketed as an appetite suppressant.

There are approximately 20 species of Hoodia including those referred to as but not limited to: H. gordonii, H. alstonii, H. currorii, H. dregei, H. flava, H. juttae, H. mossamedensis, H. officinalis, H. parviflora, H. pedicellata, H. pilifera, H. ruschii and H. triebneri.

It is herein understood by the inventors that inclusion of Hoodia in a composition will support the maintenance or reduction of body weight in a subject by reducing appetite. Embodiments of the present disclosure may contain Hoodia plant material. The preferred species of Hoodia is H. gordonii. The preferred form of Hoodia plant material is the whole plant not including the roots.

Cissus quadrangularis

Cissus quadrangularis is a plant indigenous to India where it is part of traditional medicine. Extracts of Cissus quadrangularis contain sterols, vitamin C, and tannins with antioxidant activity. The antioxidant activity has been proposed to be a mechanism of protection against tissue injury. In human studies Cissus quadrangularis has been shown to have efficacy as a weight loss agent, associated with lower cholesterol, low density lipoproteins and fasting blood glucose levels, as well as increased high density lipoproteins, the levels of which have may be inversely correlated with body mass index. The mechanism by which Cissus quadrangularis acts within this context may be, at least in part, due to relatively high antioxidant activity that may alleviate oxidative stress induced by obesity. Cissus quadrangularis has also demonstrated inhibitory action for pancreatic lipase, alpha-amylase and alpha-glucosidase, as well as the effect of increasing levels of serotonin leading to reduced appetite.

It is herein understood by the inventors that inclusion of Cissus quadrangularis in a composition will support the maintenance or reduction of body weight in a subject by at least one of providing antioxidants, increasing high density lipoproteins, lowering blood glucose levels, inhibiting lipase, inhibiting alpha-amylase, inhibiting alpha-glucosidase and increasing serotonin. Embodiments of the present disclosure may contain Cissus quadrangularis plant material. The preferred form of Cissus quadrangularis plant material is a stem and leaf extract. The most preferred Cissus quadrangularis stem and leaf extract is standardized for about 2.5% phytosterols.

Methylxanthines

Methylxanthines are methylated forms of the purine base, xanthine, which is found throughout most tissues and fluids of various organisms. Common methylxanthine derivatives include caffeine, paraxanthine, theophylline, and theobromine. Methylxanthine derivatives are nonselective adenosine receptor antagonists, central nervous system stimulants, and are often used to ward off drowsiness and restore alertness. Methylxanthine derivatives, most commonly caffeine, are widely used to control weight through multiple mechanisms including increased lipolysis of fat and increased energy expenditure, or thermogenesis.

It is herein understood by the inventors that inclusion of methylxanthines in a composition will support the maintenance or reduction of body weight in a subject by increasing energy expenditure through thermogenesis and/or increasing fat lipolysis. In one embodiment, which is set forth in greater detail in the examples below, the composition comprises caffeine. A serving of the composition according to this embodiment comprises from about 1 mg to about 400 mg of caffeine. A serving of the composition of this embodiment preferably comprises about 200 mg of caffeine, preferably caffeine anhydrous.

Morus

The genus Morus refers to an edible plant, commonly known as mulberry, which is rich in flavonoids with antioxidant activity and has long been a staple of Chinese medicine. There are over 150 species names that have been published for Mulberry. However, the following are the species that are generally accepted: alba, australis, celtidifolia, insignis, mesozygia, microphylla, nigra, and rubra.

Oral administration of mulberry, preferably Morus alba, leaves have been shown to result in weight loss and reduced increases in glucose levels after eating, which has been linked to reduced carbohydrate absorption. Furthermore, carbohydrate absorption has been shown to be attenuated, in healthy humans, by tea extracts comprising mulberry. The malabsorption of carbohydrates has been attributed to specific constituents of mulberry tea extract that inhibit alpha-glucosidase activity.

It is herein understood by the inventors that inclusion of extracts of Morus, particularly extracts of Morus alba, in a composition will support the maintenance or reduction of body weight in a subject by inhibiting the uptake of ingested carbohydrates from the gastrointestinal tract by inhibition of gluosidases. Inhibition of glucosidases in the gastrointestinal tract causes a decline in carbohydrate degradation necessary for absorption, thus leading to decreased absorption of carbohydrates. In one embodiment, which is set forth in greater detail in the examples below, the composition comprises an extract of Morus sp., preferably an extract of Morus alba leaves. A serving of the composition according to this embodiment comprises from about 0.1 g to about 2.0 g of an extract of Morus sp. In the preferred dosage of the present invention, a serving of the composition comprises about 1.2 g of an extract of Morus sp., preferably an extract of Morus alba leaves.

Chromium Polynicotinate

Chromium is an essential trace mineral that is used to control blood sugar levels by aiding insulin, which can help control or reduce weight. Chromium, as it is poorly absorbed by the body, must be combined with a more efficiently absorbed compound such as niacin, typically multiple niacin molecules, i.e. polynicotinate. Chromium likely exerts its main function as a component of the glucose tolerance factor, which is involved in insulin sensitivity. Increased levels of glucose tolerance factor have been linked to decreased serum glucose levels and free fatty acids in blood. Niacin-bound chromium has been shown clinically to spare lean mass and reduce body fat. Furthermore, chromium has also been shown to increase high density lipoprotein.

It is herein understood by the inventors that inclusion of chromium polynicotinate in a composition will support maintenance or reduction of body weight in a subject by facilitating an increase in glucose tolerance factor levels. Increased levels of glucose tolerance factor act to reduce levels of glucose and free fatty acids in the serum. In one embodiment, which is set forth in greater detail in the examples below, the composition comprises chromium polynicotinate. A serving of the composition according to this embodiment comprises from about 0.1 mg to about 5 mg of chromium polynictotinate. In the preferred dosage of this embodiment, a serving of the composition comprises about 1.33 mg of chromium polynicotinate.

Hydroxycitric Acid

Hydroxycitric acid (HCA) may be extracted from the fruits of the Garcinia Cambogia, Garcinia indica, and Garcinia atrovirdis plants. HCA is used to control weight by virtue of its ability to inhibit fat production and suppress appetite.

U.S. patent application No. 10/425,428, entitled “Hydroxycitric acid salt composition and method of making” describes a method for producing stable and pure calcium and potassium hydroxycitric acid salts which are essentially tasteless. The hydroxycitric acid salts are prepared by a process that includes treating an aqueous extract of Garcinia cambogia with a liquid quaternizing agent. The extract is further treated with potassium hydroxide or sodium hydroxide. Potassium hydroxycitrate can then be recovered and the sodium solution is further treated with a calcium salt to yield a recoverable calcium hydroxycitrate.

HCA has been shown to inhibit fatty acid synthesis and repress appetite. It is known to be a competitive inhibitor of ATP: citrate lyase; an enzyme necessary for the conversion of carbohydrates into fat. By inhibiting this enzyme, HCA blocks the body's ability to produce fat from carbohydrate sources. Human clinical trials have demonstrated the safety and beneficial effects of HCA in terms of weight management.

It is herein understood by the inventors that inclusion of hydroxycitric acid or derivatives of hydroxycitric acid in a composition will support maintenance or reduction of body weight in a subject by acting to inhibit fatty acid synthesis and curb or control appetite. By inhibiting the enzyme responsible for converting excess carbohydrates into fat, HCA acts to reduce body fat accumulation resulting from ingestion of excess calories. In one embodiment, which is set forth in greater detail in the examples below, the composition comprises an effective amount of hydroxycitric acid. A serving of the composition according to this embodiment comprises from about 0.50 g to about 3.0 g of hydroxycitric acid. In the preferred dosage of the present invention, a serving of the composition comprises about 0.933 g of hydroxycitric acid, particularly from an extract of Garcina cambogia.

Flavanols

Flavanols are a subgroup of a class of plant secondary metabolites often collectively referred to as flavonoids or bioflavonoids. They are found widely throughout nature, most often concentrated in fruits, vegetables, wines, teas and cocoa. Flavonoids have been shown to have anti-allergic, anti-inflammatory and anti-cancer activity. Flavonoids are present in a large number of plants for various purposes ranging from pigmentation to protection from attack by microbes and insects.

Substances, such as green tea, which are known to contain large amounts of various flavanols, have been shown to be effective in aiding weight loss. This effect may be due to two activities. Green tea both reduces fat digestion and increases energy expenditure. Fat stores may provide the energy necessary for the increase in energy expenditure via the oxidation of fat, consequently leading to thermogenesis. The thermogenic activity of green tea may additionally be greatly enhanced by synergistic cooperation with additionally added caffeine. In the regard of the β-oxidation of fats owing to green tea is a result of activities at adenosine receptors and their effect on an increase in cyclic adenosine monophosphate (cAMP).

Additionally, the mechanism of action of green tea may also be, due to an increase in norepinephrine. Flavanols, i.e. catechins, found in green tea, are known to inhibit catechol-O-methyl-transferase (COMT), an enzyme which degrades norepinephrine. In turn, norepinephrine inhibits the degradation as well as increases the production of cyclic adenosine monophosphate (cAMP). Furthermore, increasing norepinephrine levels by the inhibition of norepinephrine uptake results in increased weight loss in both lean and obese mice as evidenced in animal studies. Stemming from increased norepinephrine levels is the result of an increased presence of CAMP which leads to greater activation of protein kinase A, which subsequently activates lipases found in adipose tissue.

It is herein understood by the inventors that inclusion of flavanols in a weight loss composition will support weight loss in a subject by concomitantly reducing fat digestion and increasing energy expenditure. Flavanols inhibit COMT, which acts to degrade norepinephrine, thereby increasing levels of norepinephrine. Norepinephrine inhibits the degradation and enhances production of CAMP.

In an embodiment of the present invention which is set forth in greater detail in the examples below, the composition comprises an effective amount of a flavanol. A serving of the composition comprises from about 100 mg to about 700 mg of a flavanol. In the preferred dosage of the present invention, a serving of the composition comprises about 200 mg of a flavanol.

In a first embodiment of the present disclosure, a composition comprising an ingredient with appetite suppressing activity is provided with another ingredient having activity conducive to maintaining or reducing body weight other than, or in addition to, appetite suppressing activity. The preferred ingredient with appetite suppressing activity is Caralluma plant material.

In a second embodiment of the present disclosure, a composition comprising more than one ingredient directed towards suppressing the appetite of a subject in addition to another ingredient for reducing or maintaining body weight with activity other than, or in addition to, suppressing the appetite is provided.

Further embodiments are provided which contain more than one ingredient for reducing or maintaining body weight with activity other than, or in addition to, suppressing the appetite in addition to one or more ingredient directed towards suppressing the appetite of a subject.

The present disclosure further provides methods of administering the disclosed compositions to subjects directed at maintaining or reducing body weight.

Although the following examples illustrate the practice of the present invention in three of its embodiments, the examples should not be construed as limiting the scope of the invention. Other embodiments will be apparent to one of skill in the art from consideration of the specifications and example.

### EXAMPLES

### Example 1

A composition for weight loss in individuals is provided as a serving of 2 caplets to be taken twice daily prior to meal time. Each 2 caplet serving may comprise:
Caralluma fimbriata and Cissus quadrangularis.

### Example 2

A composition is provided as in Example 1, wherein each 2 caplet serving comprises:
Caralluma fimbriata extract (aerial parts) and Cissus quadrangularis.extract (stem and leaves).

### Example 3

A composition is provided as in Example 1, wherein each 2 caplet serving comprises:
from about 1 mg to about 500 mg Caralluma fimbriata extract (aerial parts) and from about 1 mg to about 500 mg Cissus quadrangularis.extract (stem and leaves).

### Example 4

A composition for weight loss in individuals is provided as a serving of 3 liquid capsules to be taken twice daily prior to meal time. Each 3 capsule serving may comprise:
Caralluma fimbriata and Hoodia gordonii.

### Example 5

A composition is provided as in Example 4, wherein each 3 capsule serving comprises:
Caralluma fimbriata extract (aerial parts) and Hoodia gordonii (whole plant less roots).

### Example 6

A composition is provided as in Example 4, wherein each 3 capsule serving comprises:
from about 1 mg to about 500 mg Caralluma fimbriata extract (aerial parts) and from about 1 mg to about 250 mg Hoodia gordonii (whole plant less roots).

### Example 7

A composition for weight loss in individuals is provided as a serving of 2 caplets to be taken twice daily prior to meal time. Each 2 caplet serving may comprise:
150 mg Cissus quadrangularis extract (stem and leaves), 100 mg Hoodia gordonii powder (whole plant less roots) and 1 mg Caralluma fimbriata extract (aerial parts).

### Example 8

A composition for weight loss in individuals is provided as a serving of 3 liquid capsules to be taken twice daily prior to meal time. Each 3 capsule serving may comprise:
450 mg Green tea extract, (Camellia sinensis—leaf) and 10 mg Caralluma fimbriata extract (aerial parts).

### Example 9

A composition for weight loss in individuals is provided as a serving of 3 liquid capsules to be taken twice daily prior to meal time. Each 3 capsule serving may comprise:
300 mg caffeine anhydrous and 10 mg Caralluma fimbriata extract (aerial parts).

Extensions and Alternatives

In the foregoing specification, the invention has been described with a specific embodiment thereof; however, it will be evident that various modifications and changes may be made thereto without departing from the broader spirit and scope of the invention.

All publications which are cited herein are hereby specifically incorporated by reference into the disclosure for the teachings for which they are cited.

What is claimed:

1. A method of maintaining or reducing the body weight of a subject by providing a composition comprising:
a) an appetite-reducing compound, and
b) a compound with activity conducive to maintaining or reducing weight other than, or in addition to, appetite-reducing activity.

2. The method of claim 1, wherein;
a) is a Caralluma species or a Hoodia species and
b) is selected from the group consisting of Cissus quadrangularis, methylxanthines, Morus alba, chromium polynicotinate, hydroxycitric acid, and flavanols.

3. A composition comprising:
a) an appetite-reducing compound, and
b) a compound with activity conducive to maintaining or reducing weight other than, or in addition to, appetite-reducing activity.

4. The composition of claim 3, wherein;
a) is a Caralluma species or a Hoodia species and
b) is selected from the group consisting of: Cissus quadrangularis, methylxanthines, Morus alba, chromium polynicotinate, hydroxycitric acid, and flavanols.

5. The composition of claim 4 wherein;
a) is Caralluma fimbriata and
b) is Cissus quadrangularis.

6. The composition of claim 4 wherein;
a) is Caralluma fimbriata and
b) is a methylxanthine.

7. The composition of claim 4 wherein;
a) is Caralluma fimbriata and
b) is chromium polynicotinate.

8. The composition of claim 4 wherein;
a) is Caralluma fimbriata and
b) is hydroxycitric acid.

9. The composition of claim 4 wherein;
a) is Caralluma fimbriata and
b) is a flavanol.

10. The composition of claim 5 wherein;
a) is Hoodia gordonii and
b) is Cissus quadrangularis.

11. The composition of claim 5 wherein;
a) is Hoodia gordonii and
b) is a methylxanthine.

12. The composition of claim 5 wherein;
a) is Hoodia gordonii and
b) is chromium polynicotinate.

13. The composition of claim 5 wherein;
a) is Hoodia gordonii and
b) is hydroxycitric acid.

14. The composition of claim 5 wherein;
a) is Hoodia gordonii and
b) is a flavanol.

15. A composition comprising Caralluma fimbriata, Hoodia gordonii, and Cissus quadrangularis.

16. The composition of claims 15, further comprising at least one of: a methylxanthine, Morus alba, chromium polynicotinate, hydroxycitric acid, or a flavanol.
